(19) United States
(12) Patent Application Publication
(10) Pub. No.: US 2006/0128720 A1
(43) Pub. Date: Jun. 15, 2006

(54) Inhibition of cell death responses induced by oxidative stress

(76) Inventors: Donald W. Kufe, Wellesley, MA (US); Rima Kaddurah-Daouk, Belmont, MA (US)

Correspondence Address: FISH & RICHARDSON P.C., P.O. BOX 1022, MINNEAPOLIS, MN, 55440-1022, US

(21) Appl. No.: 10/518,665

(22) PCT Filed: Mar. 20, 2003
(86) PCT No.: PCT/US03/10112

§ 371 Date: Nov. 7, 2005

Related U.S. Application Data
(60) Provisional application No. 60/366,410, filed on Mar. 21, 2002.

(51) Int. Cl. A61K 31/506

(52) U.S. Cl. 514/252.18

(57) ABSTRACT

The invention provides methods of reducing or preventing oxidative stress-induced cell death by contacting a cell with a compound that inhibits the kinase activity and/or the mitochondrial translocation of c-Abl. The methods of the invention can be used to treat individuals individual diagnosed as having or being at risk of contracting a disorder characterized by excessive oxidative stress-induced cell death.

### CROSS REFERENCE TO RELATED APPLICATIONS

This application claims priority from U.S. Provisional Application No. 60/366,410, filed Mar. 21, 2002, the content of which is incorporated herein by reference in its entirety.

### STATEMENT AS TO FEDERALLY SPONSORED RESEARCH

This invention was made with Government support under grant number CA42802 awarded by the National Cancer Institute. The Government may have certain rights in the invention.

### FIELD OF THE INVENTION

The invention relates to methods of abrogating cell death responses associated with oxidative stress.

### BACKGROUND OF THE INVENTION

Normal cellular metabolism is associated with the production of reactive oxygen species (ROS) and, as a consequence, damage to DNA and proteins. ROS have been implicated as signaling molecules that contribute to neurodegenerative diseases and aging. The generation of ROS is associated with apoptosis and necrotic cell death. Certain cells, particularly neurons, are highly sensitive to ROS-induced apoptosis. Studies have indicated that ROS-induced apoptosis is p53-dependent and that p53-induced apoptosis is mediated by ROS. In addition, the p66shc adaptor protein and the p85 subunit of phosphatidylinositol 3-kinase (PI3-K) have been implicated in the apoptotic response to oxidative stress.

The ubiquitously expressed c-Abl protein tyrosine kinase localizes to the nucleus and cytoplasm. The nuclear form of c-Abl is activated in the cellular response to genotoxic stress (Kharbanda et al. (1995) Nature 376:785-88) and contributes to the induction of apoptosis by mechanisms in part dependent on p53 and its homolog p73 (Yuan et al. (1996) Nature 382:272-74). The cytoplasmic form of c-Abl is activated in response to oxidative stress by a mechanism dependent on protein kinase C delta (Sun et al. (2000) J. Biol. Chem. 275:17237-40; Sun et al. (2000) J. Biol. Chem. 275:7470-73). Targeting of c-Abl to mitochondria is associated with the cell death response to oxidative stress (Kumar et al. (2001) J. Biol. Chem. 17281-85).

### SUMMARY OF THE INVENTION

The invention is based on the discovery that small molecule inhibitors of the tyrosine kinases c-Abl and/or Arg (nonreceptor tyrosine kinase that has an overall structure similar to that of c-Abl) can be used to prevent cell death associated with oxidative stress.

Normal aerobic metabolism is associated with the production of reactive oxygen species (ROS) and, as a consequence, damage to DNA and proteins. The apoptotic and necrotic responses to oxidative stress are thought to contribute to disorders such as neurological degeneration as well as the aging process. STI571, an inhibitor of the Bcr-Abl oncoprotein in chronic myelogenous leukemia, was found to block the activation of c-Abl in the cellular response to oxidative stress. As detailed in the Examples, immunofluorescence microscopy and subcellular fractionation analyses demonstrated that STI571 abrogates H₂O₂-induced targeting of c-Abl to mitochondria and attenuates H₂O₂-induced loss of mitochondrial transmembrane potential. In addition, STI571 was found to exhibit a substantial inhibitory effect on the apoptotic response to H₂O₂ exposure. These findings indicate that STI571 inhibits, at least in part, ROS-induced mitochondrial dysfunction and the apoptotic response to oxidative stress.

STI571 (also known as CGP57148B, imatinin mesylate, and Gleevec™) inhibits the tyrosine kinase activities of Bcr-Abl, c-Abl, platelet-derived growth factor receptor and c-kit, but is inactive against other tyrosine and serine/threonine kinases (Druker et al. (1996) Nature Med. 2:561-66; and Carroll et al. (1997) Blood 90:4947-52). STI571 is highly effective in the treatment of chronic myelogenous leukemia and induces the apoptosis of chronic myelogenous leukemia cells in culture (Druker et al. (1996) Nature Med. 2:561-66; and Druker et al. (2001) N. Eng. J. Med. 344:1038-42). In contrast, the experimental data presented herein demonstrate that STI571 inhibits ROS-induced mitochondrial dysfunction and apoptosis. The finding that STI571 prevents apoptosis was unexpected in light of the molecule's prior characterization as an effective inducer of apoptosis in cells derived from patients having chronic myelogenous leukemia

In one aspect, the invention features a method of reducing or preventing oxidative stress-associated cell death. The method includes the steps of: selecting an individual diagnosed as having or being at risk of contracting a disorder characterized by excessive oxidative stress-associated cell death; and administering to the individual a composition containing an N-phenyl-2-pyrimidine-amine described herein in an amount effective to reduce or prevent oxidative stress-associated cell death in the individual.

“Oxidative stress” refers to the generation of reactive oxygen species (ROS) within a cell. Examples of ROS include singlet oxygen, hydroxyl radicals, superoxide, hydroperoxides, and peroxides.

“Oxidative stress-associated cell death” refers to necrotic and/or apoptotic cell death that occurs following exposure of a cell to ROS.

The N-phenyl-2-pyrimidine-amine can be, for example, 4-[(4-Methyl-1-piperazinyl)methyl]-N-[4methyl-3-[[4(3-pyridinyl)-2-pyrimidinyl]amino-]-phenyl]benzamide methanesulfonate.

In one embodiment, the individual has been diagnosed as having a disorder characterized by excessive oxidative stress-associated cell death. In another example, the individual has been diagnosed as being at risk of contracting a disorder characterized by excessive oxidative stress-associated cell death.

In one embodiment, the individual has been diagnosed as having a neurological disorder, e.g., Alzheimer's disease, Parkinson's disease, Huntington's disease, amyotrophic lateral sclerosis, multiple sclerosis, retinitis pigmentosa, or spinal muscular atrophy.

In some embodiments, e.g., where the individual has been diagnosed as having a neurological disorder, the methods further include administering to the individual a second therapeutic compound, wherein the second therapeutic compound reduces or prevents symptoms of the neurological disorder. Examples of a second therapeutic compound include riluzole, tacrine, donepizil, carbidopa/levidopa, carbidopa/levidopa sustained release, pergolide mesylate, bromocriptine mesylate, selgiline, amantadine, or trihexyphenidyl hydrochloride. In some examples, the second therapeutic compound is a dopamine receptor antagonist. In other examples, the second therapeutic compound is a glutamate excitotoxicity inhibitor, growth factor, nitric oxide synthase inhibitor, cyclo-oxygenase inhibitor, ICE inhibitor, neuroimmunophilin, N-acetylcysteine, procysteine, antioxidant, or lipoic acid.

In some embodiments, the methods further include a step of carrying out a neurological test on the individual after administering the composition to the individual. In some embodiments, the methods include again administering the composition to the individual after carrying out the neurological test, wherein the amount of the composition administered in the second administration is determined at least in part based upon results obtained from the neurological test.

In some embodiments, the individual has been diagnosed as being at risk of contracting a neurological disorder, e.g., Alzheimer's disease, Parkinson's disease, Huntington's disease, amyotrophic lateral sclerosis, multiple sclerosis, retinitis pigmentosa, or spinal muscular atrophy.

In some embodiments the disorder characterized by excessive oxidative stress-associated cell death is caused by an ischemia/reperfusion injury. For example, in some cases, the individual has: been diagnosed as having had a myocardial infarction or stroke; undergone or is undergoing an organ transplant surgery; or undergone or is undergoing coronary bypass surgery.

The methods also include administering to the individual a second therapeutic compound, wherein the second therapeutic compound reduces or prevents symptoms of the disorder characterized by excessive oxidative stress-associated cell death caused by an ischemia/reperfusion injury. In some examples, the second therapeutic compound is a thrombolytic or an anticoagulant.

In some embodiments, the method further includes a step of carrying out a test for ischemia/reperfusion injury on the individual after administering the composition to the individual. In addition, the methods can further include a step of again administering the composition to the individual after carrying out the test for ischemia/reperfusion injury, wherein the amount of the composition administered in the second administration is determined at least in part based upon results obtained from the test for ischemia/reperfusion injury.

In some embodiments, the individual has been diagnosed as having an inflammatory disorder, e.g., arthritis.

In some embodiments, the methods further include a step of evaluating the viability of a neurological or cardiovascular tissue of the individual following the administration of the composition to the individual.

The composition can be administered to the individual by a variety of routes as described herein, e.g., by injection or via a catheter.

In some embodiments of the methods described herein, the individual has not been diagnosed as having a cancer, e.g., a hematological cancer. For example, the invention includes methods wherein the individual has not been diagnosed as having chronic myelogenous leukemia.

In another aspect, the invention features a method of preventing or reducing cell death in a cell population. The method includes the steps of: providing a cell population; and contacting the cell population with a composition comprising an N-phenyl-2-pyrimidine-amine described herein in an amount effective to prevent or reduce cell death in the cell population. The method can be carried out on a cell population in vivo or in vitro. The method can include an additional step of detecting the effectiveness of the composition at preventing or reducing cell death in the cell population.

The N-phenyl-2-pyrimidine-amine can be, for example, 4-[(4-Methyl-1-piperazinyl)methyl]-N-[4methyl-3-[[4(3-pyridinyl)-2-pyrimidinyl]amino-]-phenyl]benzamide methanesulfonate.

In some embodiments, the method also includes a step of determining the viability of the cell population, wherein the viability of the cell population is increased as compared to the viability predicted in the absence of contacting the cell population with the composition.

In some embodiments, the cell population does not include cancer cells.

In some embodiments, the cell population does not include chronic myelogenous leukemia cells.

In some embodiments, the cell population contains neural cells.

In some embodiments, the cell population contains cells that have undergone an ischemia/reperfusion injury.

In another aspect, the invention features a method of reducing or preventing aging-related cellular degeneration in an individual by administering to the individual a composition containing an N-phenyl-2-pyrimidine-amine described herein in an amount effective to reduce or prevent aging-related cellular degeneration in the individual. The method can include a step of detecting the effectiveness of the composition at preventing or reducing aging-related cellular degeneration in the individual.

The N-phenyl-2-pyrimidine-amine can be, for example, 4-[(4-Methyl-1-piperazinyl)methyl]-N-[4methyl-3-[[4(3-pyridinyl)-2-pyrimidinyl]amino-]-phenyl]benzamide methanesulfonate.

In some embodiments, the individual has not been diagnosed as having a cancer, e.g., a hematological cancer.

In some embodiments, the individual has not been diagnosed as having chronic myelogenous leukemia.

In another aspect, the invention features a kit containing a composition containing an N-phenyl-2-pyrimidine-amine described herein and written instructions for use to reduce or prevent aging-related cellular degeneration or treat or prevent a disorder characterized by excessive oxidative stress-associated cell death

The N-phenyl-2-pyrimidine-amine can be, for example, 4-[(4-Methyl-1-piperazinyl)methyl]-N-[4methyl-3-[[4(3-pyridinyl)-2-pyrimidinyl]amino-]-phenyl]benzamide methanesulfonate.

In some embodiments, the written instructions are for use to treat or prevent a neurological disorder, e.g., a neurological disorder described herein.

In other embodiments, the written instructions are for use to treat or prevent a disorder caused by an ischemia/reperfusion injury, e.g., a disorder described herein.

In another aspect, the invention features a pharmaceutical composition containing an N-phenyl-2-pyrimidine-amine described herein and a second therapeutic compound that is effective for the treatment of a disorder characterized by excessive oxidative stress-associated cell death.

The N-phenyl-2-pyrimidine-amine can be, for example, 4-[(4-Methyl-1-piperazinyl)methyl]-N-[4methyl-3-[[4(3-pyridinyl)-2-pyrimidinyl]amino-]-phenyl]benzamide methanesulfonate.

In one embodiment, the disorder is a neurological disorder, e.g., a neurological disorder described herein. The second therapeutic compound can be a compound described herein as useful for the treatment of a neurological disorder.

In another embodiment, the disorder is caused by an ischemia/reperfusion injury, e.g., a disorder described herein. The second therapeutic compound can be a compound described herein as useful for the treatment of a disorder is caused by an ischemia/reperfusion injury.

STI571 has been successfully used as a therapeutic agent in patients having chronic myelogenous leukemia, a hematological cancer characterized by insufficient apoptosis of cells expressing the oncoprotein Bcr-Abl. STI571 to has been found to induce apoptosis of Bcr-Abl-expressing cells and to provide a therapeutic benefit to patients having chronic myelogenous leukemia Accordingly, STI571 has been understood to be useful in treating proliferative diseases, such as cancer, that are characterized by insufficient apoptosis. As described in the present application, it has been unexpectedly found that STI571 can prevent cell death induced by oxidative stress. Accordingly, the compounds described herein can be used to treat a variety of disorders characterized by excessive cell death.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is an autoradiograph depicting the inhibition of c-Abl kinase activity by H₂O₂. Mouse embryo fibroblasts were pretreated with the indicated concentrations of STI571 for 24 hours and then treated with 1 mM H₂O₂ for 15 minutes. Cell lysates were subjected to immunoprecipitation (IP) with anti-c-Abl. The immunoprecipitates were incubated with [γ-³²P]ATP and GST-Crk(120-225) (upper panel) or [γ-³²P]ATP and GST-Crk(120-212) (middle panel) for 20 minutes. The reaction products were analyzed by SDS-PAGE and autoradiography. The immunoprecipitates were also subjected to immunoblotting (IB) with anti-c-Abl (lower panel).

FIGS. 2A-2B are graphs that depict the attenuation by STI571 of the loss of mitochondrial transmembrane potential in response to H₂O₂. Mouse embryo fibroblasts (A) and U-937 cells (B) were treated with 10 mM STI571 for 24 hours and then exposed to 1 mM H₂O₂ for 6 hours. The cells were stained with 50 ng/ml Rhodamine123 for 15 minutes and analyzed by flow cytometry (left panels). The percentage (mean±S.E.) of control mitochondrial transmembrane potential was determined after treatment with H₂O₂ and STI571 (black bars) and after treatment with H₂O₂ alone (white bars) (right panels).

FIGS. 3A-3D are graphs that depict the inhibition by STI571 of the apoptotic response to oxidative stress. Mouse embryo fibroblasts, (A) and (B), and U-937 cells, (C) and (D), were treated with 10 mM STI571 for 24 hours and then exposed to 1 mM H₂O₂ for 18 hours. Ethanol-fixed cells were stained with propidium iodide and monitored for sub-G1 DNA (upper panels). The percentage (mean±S.E.) of cells with sub-G1 DNA was determined from three separate experiments (lower panels).

### DETAILED DESCRIPTION

The present invention provides methods of reducing or preventing oxidative stress-associated cell death in an individual diagnosed as having or being at risk of contracting a disorder characterized by excessive oxidative stress-associated cell death. As described in the accompanying examples, the compound STI571 was found to inhibit H₂O₂-induced targeting of c-Abl to mitochondria, attenuate H₂O₂-induced loss of mitochondrial transmembrane potential, and inhibit the apoptotic response following H₂O₂ exposure. Accordingly, the methods and compositions of the invention are directed to the reduction or prevention of oxidative stress-associated cell death in a manner similar to that observed with STI571.

### Pharmaceutical Compositions

As described herein, compounds that inhibit the kinase activity and/or the mitochondrial translocation of c-Abl and/or or Arg can be used to prevent or reduce cell death caused by oxidative stress. A wide variety of compounds can be used to inhibit c-Abl and/or or Arg activities. In vitro and in vivo assays described in the following sections can be used to confirm the ability of a compound to inhibit an activity of c-Abl and/or Arg and/or to inhibit cell death caused by oxidative stress.

Compounds that can be used according to the methods described herein include N-phenyl-2-pyrimidine-amine derivatives such as those described in U.S. Pat. No. 5,521,184. Any of the compounds described in U.S. Pat. No. 5,521,184, the entire contents of which are incorporated by reference, that inhibit the kinase activity and/or mitochondrial translocation of c-Abl and/or or Arg can be used in the methods of the invention.

In some embodiments, a compound used in a method of the invention is an N-phenyl-2-pyrimidine-amine compound of formula I

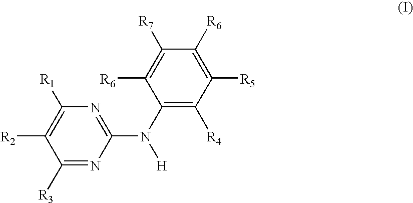

wherein

R₁ is 4-pyrazinyl, 1-methyl-1H-pyrrolyl, amino-, or amino-lower alkyl-substituted phenyl, wherein the amino group in each case is free, alkylated or acylated, 1H-indolyl or 1H-imidazolyl bonded at a five-membered ring carbon atom, or unsubstituted or lower alkyl-substituted pyridyl bonded at a ring carbon atom and unsubstituted or substituted at the nitrogen atom by oxygen,

R₂ and R₃ are each, independently of the other, hydrogen or lower alkyl;

one or two of the radicals R₄, R₅, R₆, R₇ and R₈ are each nitro, fluoro-substituted lower alkoxy or a radical of formula II
—N(R₉)—C(═X)—(Y)ₙ—R₁₀ (II)

wherein

R₉ is hydrogen or lower alkyl,

X is oxo, thio, imino, N-lower alkyl-imino, hydroximino or O-lower alkyl-hydroximino,

Y is oxygen or the group NH,

n is 0 or 1 and

R₁₀ is an aliphatic radical having at least 5 carbon atoms, or an aromatic, aromatic-aliphatic, cycloaliphatic, cycloaliphatic-aliphatic, heterocyclic or hetero-cyclicaliphatic radical,

and the remaining radicals R₄, R₅, R₆, R₇ and R₈ are each, independently of the others, hydrogen, lower alkyl that is unsubstituted or substituted by free or alkylated amino, piperazinyl, piperidinyl, pyrrolidinyl or morpholinyl, or lower alkanoyl, trifluoromethyl, free, etherified or esterifed hydroxy, free, alkylated or acylated amino or free or esterified carboxy,

or a salt of such a compound having at least one salt-forming group.

In some embodiments, a compound used in a method of the invention is of the formula

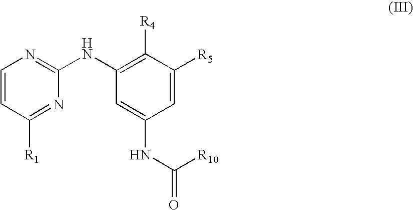

wherein,

R₁ is pyridyl,

R₄ is methyl,

R₅ is hydrogen, and

Rᵢₛ 4-methyl-piperazinylmethyl.

An example of a useful compound that can be used in the methods described herein is STI571 (signal transduction inhibitor number 571). STI571 is a rationally developed, selective tyrosine kinase inhibitor that has been used as a therapeutic agent to treat patients having chronic myelogenous leukemia STI571 is described in detail in, e.g., Mauro et al. (2001) Oncologist 6:233-238, the contents of which are incorporated by reference.

STI571 is designated chemically as 4-[(4-Methyl-1-piperazinyl)methyl]-N-[4-methyl-3-[[4-(3-pyridinyl)-2-pyrimidinyl]amino-]-phenyl]benzanide methanesulfonate. STI571 and related compounds are described in detail in WO 01/47507, EP 0564409, and WO 99/03854, the entire contents of which are incorporated by reference. Any of the compounds described by these references can potentially be used in the methods of the invention.

The chemical structure of STI571 is as follows:

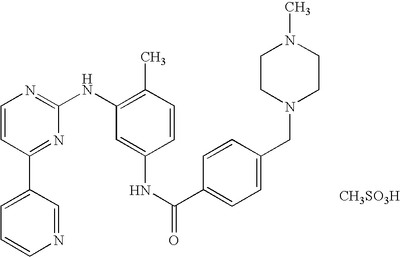

In addition to the compounds described in the references mentioned above, those compounds described in U.S. Pat. No. 6,306,874, WO 97/02266, and WO 98/35958 that inhibit c-Abl and/or Arg kinase activity and/or mitochondrial translocation of c-Abl and/or Arg can also be used in the methods of the invention. The entire content of each of these references is incorporated by reference.

Compounds described herein can be formulated so as to facilitate their crossing of the blood brain barrier when administered to an individual.

In those embodiments of the invention that involve the administration of a compound described herein to an animal, e.g., a human, the compound can be formulated in a pharmaceutical composition. Pharmaceutical compositions for use in accordance with the present invention can be formulated in a conventional manner using one or more physiologically acceptable carriers or excipients. Thus, the compounds and their physiologically acceptable salts and solvates may be formulated for administration by inhalation (either through the mouth or the nose), oral, buccal, parenteral or rectal administration. Preferred methods of parenteral administration include intravenous, intraarterial, intramuscular, subcutaneous, subdermal, intradermal, intraperitoneal, and intrathecal administration.

Excipients that can be used in a formulation of a compound described herein include buffers (e.g., citrate buffer, phosphate buffer, acetate buffer, and bicarbonate buffer), amino acids, urea, alcohols, ascorbic acid, phospholipids, proteins (e.g., serum albumin), EDTA, sodium chloride, liposomes, mannitol, sorbitol, and glycerol.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The compounds may be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions, or emulsions in oily or aqueous vehicles, and may contain formulatory agents such as suspending, stabilizing and/or dispersing agents. Alternatively, the active ingredient can be in powder form for constitution with a suitable vehicle, for example, sterile pyrogen-free water, before use.

The compounds can also be formulated in rectal compositions such as suppositories or retention enemas containing, for example, conventional suppository bases such as cocoa butter or other glycerides.

In addition to the formulations described previously, the compounds may also be formulated as a depot preparation. Such long acting formulations may be administered by implantation (e.g., subcutaneously or intramuscularly) or by intramuscular injection. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

Methods of administering the compounds described herein can be optimized to promote the ability of the compound to cross the blood-brain barrier. In one example, a compound can be directly administered to the cerebrospinal fluid, e.g., by intraventricular injection. In another example, it may be desirable to administer a compound locally to an area or tissue in need of treatment. This may be achieved, for example, by local infusion during surgery, by topical application, by injection, by infusion using a cannulae with osmotic pump, by means of a catheter, by means of a suppository, or by means of an implant. Examples of tissues to which a compound can be administered according to any of these methods include cardiovascular tissues and neurological tissues.

The compositions described herein can be administered to an individual in an amount effective to reduce or prevent oxidative stress-associated cell death in the individual. For example, effective doses can range from 20-5,000 mg/day, 100-1,000 mg/day, 200-800 mg/day, or 400-600 mg/day. Of course, as is well known in the medical arts, dosage for any given individual depends upon many factors, including the patient's size, body surface area, age, the particular compound to be administered, sex, time and route of administration, general health, and other drugs being administered concurrently. Determination of optimal dosage is well within the abilities of a pharmacologist of ordinary skill.

Disorders Associated with Cell Death Induced by Oxidative Stress

As described herein, compounds such as STI571 that inhibit c-Abl and/or Arg kinase activity and/or mitochondrial translocation of c-Abl and/or Arg can be used to treat an individual diagnosed as having or being at risk of contracting a disorder characterized by excessive oxidative stress-associated cell death. Cell death induced by oxidative stress includes necrotic cell death and/or apoptotic cell death. The following is a non-limiting description of disorders that are characterized by excessive oxidative stress-associated cell death that can be treated using the compositions and methods described herein.

A wide variety of neurological diseases are characterized by the gradual loss of neurons. It is disorders such as these, associated with excessive levels of necrotic and/or apoptotic cell death, to which the compositions and methods of the invention can be applied. Such disorders include neuropathies (e.g., diabetic and toxic neuropathies), motor neuron disease, traumatic nerve injury, acute disseminated encephalomyelitis, acute necrotizing hemorrhagic leuokoencephalitis, dysmyelination disease, peripheral nervous system diseases, and various forms of cerebellar degeneration. Examples of specific neurological diseases that can be treated using the compounds described herein include Alzheimer's disease, Parkinson's disease, Huntington's disease, amyotrophic lateral sclerosis, multiple sclerosis, retinitis pigmentosa, and spinal muscular atrophy.

The compositions and methods of the invention can also be used to treat disorders associated with ischemia/reperfusion injury. Such disorders can result from a vascular disease and/or a surgical procedure carried out on an individual. For example, in the course of some surgical procedures (e.g., coronary bypass surgery or organ transplantation surgery such as kidney or heart transplant surgery) blood vessels are intentionally occluded to allow a surgeon free access to the surgical site. The occlusion is subsequently removed and reperfusion of blood (to a surgical site or a transplanted organ) occurs. In addition, some patients with coronary or peripheral vascular disease are exposed to regular ischemia/reperfusion cycles (e.g., angina pectoris or intermittent claudication) that terminate spontaneously or in response to administered drugs.

Reperfusion can lead to an array of biochemical events that culminate in oxidative damage to cell structures, thereby inducing tissue damage. For example, ischemic tissue prior to reperfusion can sometimes be essentially undamaged, but often experiences massive, irreversible degradation such as cell death and tissue necrosis upon reperfusion. Reperfusion injury can occur over a period of several days time after reperfusion is begun. The compositions described herein can be used to reduce or prevent ischemia/reperfusion injury in a tissue at risk of undergoing such injury. The methods include, for example, administering a composition described herein to an individual (e.g., at a specific site in the individual at risk of undergoing such injury) as well as exposing an organ or tissue ex vivo, prior to transplantation, to a compound described herein. Accordingly, an organ or a tissue can be perfused with a composition described herein to reduce or prevent ischemia/reperfusion injury to the organ or tissue. An organ can also be perfused in situ in a donor prior to its transplantation into a recipient.

Two common disorders associated with cell death are myocardial infarctions and stroke. In both disorders, cells within a central area of ischemia, which is produced in the event of acute loss of blood flow, appear to die rapidly as a result of necrosis. In addition, cells outside the central ischemic zone die over a more protracted time period and morphologically appear to die by apoptosis. The methods and compositions described herein can be used to reduce or prevent the cell death associated with myocardial infarctions, stroke, embolisms, and other cardiovascular disorders.

In addition, the compositions and methods described herein can be used to treat other disorders characterized by excessive oxidative stress-associated cell death, including some hematological diseases, neuromuscular diseases, inflammatory diseases (e.g., arthritis), and dementia The compositions and methods described herein can also be applied to counteract the sequelae of the aging process, as aging is associated with increased oxidative damage and impaired mitochondrial functions. Mitochondria are involved in the production of reactive oxygen species, and cells having impaired mitochondrial functions are highly susceptible to oxidative stress-associated apoptotic cell death.

The invention also encompasses prophylactic methods, wherein a compound described herein is administered to an individual diagnosed as being at risk of contracting a disorder characterized by excessive oxidative stress-associated cell death. Such methods are particularly useful in cases of familial disorders, where an individual can be predicted to be especially susceptible of developing a disease before onset of symptoms occur. Examples of familial disorders include some forms of amyotrophic lateral sclerosis, Huntington's disease, and Alzheimer's disease. Methods described herein can also include steps of genetic screening to identify an individual as having a genetic profile associated with a familial disorder described herein.

In a situation such as familial amyotrophic lateral sclerosis or Huntington's disease, it may be particularly advantageous to administer a composition described herein before the onset of symptoms. For example, an individual can be diagnosed as having a mutation in the superoxide dismutase 1 gene that is associated with the development of amyotrophic lateral sclerosis. By administering a composition described herein to such an individual before the onset of symptoms, the treatment can delay the onset of symptoms and/or reduce the severity of symptoms when they do occur. In addition, such treatment can be used to extend the expected lifespan of an individual diagnosed as being susceptible to developing familial amyotrophic lateral sclerosis.

Co-Administration of Other Therapeutic Compounds

The compounds described herein can be co-administered to an individual in combination with one or more other therapeutic compounds useful for treating a disorder characterized by excessive oxidative stress-associated cell death.

In those cases where the individual has (or is at risk of having) a neurological disease, including but not limited to the specific neurological diseases described herein, a neuroprotective agent can be co-administered to the individual together with a composition described herein. Examples of neuroprotective agents include riluzole (Rilutek®), tacrine (Cognex®), donepizil (Aricept®), carbidopa/levidopa (Sinemet®), carbidopa/levidopa sustained release (Sinemet® CR), pergolide mesylate (Permax®), bromocriptine mesylate (Parlodel®), selgiline (Elepryl®), amantadine (Symmetrel®), trihexyphenidyl hydrochloride (Artane®), glutamate excitotoxicity inhibitors, growth factors (e.g., CNTF, BDNF, and IGF-1), nitric oxide synthase inhibitors, cyclo-oxygenase inhibitors (e.g., aspirin), ICE inhibitors, neuroimmunophilins, N-acetylcysteine, procysteine, antioxidants, energy enhancers, vitamins and cofactors (e.g., spin traps, CoQ10, carnitine, nicotinamide, Vitamin C, Vitamin D, and Vitamin E), and lipoic acid. Several of the neuroprotective agents include in this list are dopamine receptor antagonists and are particularly useful for the treatment of Parkinson's disease.

Examples of antioxidants include, but are not limited to, sodium metabisulfite, sodium thiosulfate, acetylcysteine, butylated hydroxyanisole, and butylated hydroxytoluene.

In those cases where the individual has (or is at risk of having) a cardiovascular disease, including but not limited to the specific cardiovascular diseases described herein, a second therapeutic compound can be co-administered to the individual together with a composition described herein. Such compounds may be anti-stroke drugs and/or may help prevent brain damage from cerebral ischemia Examples of useful therapeutic compounds include thrombolytics, anticoagulants, glutamate release inhibitors, calcium influx blockers, and NMDA receptor antagonists.

Thrombolytics include APSAC, plasmin, urokinase, pro-urokinase, streptokinase, and tissue plasminogen activator.

Heparin is an example of an anticoagulant.

In addition to the specific therapeutic compounds listed above, the methods of the invention encompass the co-administration to an individual, together with a compound described herein such as STI571, of any other therapeutic compound that can be used to treat one or more of the disorders described herein.

A therapeutic compound can be co-administered to an individual in the form of a pharmaceutical composition and using a route of administration as described herein. The co-administration of the therapeutic compound can be initiated concurrently with the administration of a compound described herein (e.g., by combining the two into a single composition). Alternatively, the therapeutic compound can be co-administered to the individual within 1, 2, 3, 4, 5, 6, 7, 8, 9, 10, 11, 12, or more hours before or after administration of a compound described herein. In other cases, the therapeutic compound is co-administered to the individual within 1, 2, 3, 4, 5, 6, 7, 14, 21, 28, or more days before or after administration of a compound described herein.

In Vitro and In Vivo Assay Systems

The compounds described herein can be used in a wide variety of in vitro and in vivo model systems. These systems can be used, for example, to confirm the bioactivity of the compounds, to establish appropriate doses, or to establish useful regimens of co-administration with a second therapeutic compound.

The compounds used in the methods of the invention inhibit c-Abl and/or Arg kinase activity and/or mitochondrial translocation of c-Abl and/or Arg. The ability of a given compound to inhibit either of these activities can be evaluated using the in vitro assays described in the accompanying examples. These assays monitor both the ability of c-Abl and/or Arg to phosphorylate a substrate upon the induction of oxidative stress and the ability of c-Abl and/or Arg to translocate to the mitochondria upon the induction of oxidative stress.

Compounds can be evaluated for their ability to reduce or prevent oxidative stress-associated cell death using a variety of in vitro assays, including the assays described in the accompanying examples. Such assays can make use of a variety of cell types, including neuronal cells and neuronal cell lines. Accordingly, many assay systems allow for the in vitro evaluation of the neuroprotective properties of a given compound. Examples of useful in vitro systems are described in detail in, e.g., Pong et al. (2001) Exp. Neurology 171:84-97; Jones et al. (2001) J. Neurochem. 74:2296-2304; Schroeter et al. (2000) Free Radical Biology & Medicine 29:1222-33; Yao et al. (2001) Brain Research 889:181-90.

The ability of a compound described herein to act as a neuroprotective agent can be evaluated in vivo. For example, compounds can be evaluated in a variety of animal model systems of neurological diseases. These animal systems can be used to evaluate the effectiveness of a compound described herein either acting on its own or when co-administered with a therapeutic agent according to the methods described herein. Examples of useful animal model systems include models of Huntington's Disease (Matthews et al. (1998) J. Neuroscience 18:156-63; and Ferrante et al. (2000) J. Neuroscience 20:4389-97), Parkinson's Disease (Matthews et al. (1999) Exp. Neurology 157:14249), amyotrophic lateral sclerosis (Klivenyi et al. (1999) Nature Medicine 5:347-50), Alzheimer's disease (Hock et al. (2001) Trends Genet.17:S7-12), and NMDA and malonate toxicity (Malcon et al. (2000) Brain Research 860:195-98).

Kits

The invention includes kits containing a composition described herein and written instructions for use to treat a disorder characterized by excessive oxidative stress-associated cell death. The disorder can be any of the disorders described herein. For example, a kit can contain a vessel containing a pharmaceutical composition comprising any N-phenyl-2-pyrimidine-amine, such as 4-[(4-Methyl-1-piperazinyl)methyl]-N-[4methyl-3-[[4(3-pyridinyl)-2-pyrimidinyl]amino-]-phenyl]benzamide methanesulfonate and written instructions to use the composition to treat one or more of the disorders described herein, e.g., a neurological disorder, an ischemia/reperfusion injury, or an inflammatory disorder. Alternatively, the instructions can specify use of the composition in storage solution and/or as a pefusate for an organ prior to transplantation in an individual.

A kit can further include a second therapeutic compound as described herein, e.g., a neuroprotective agent. The kit can also include written instructions to administer the second therapeutic compound together with the pharmaceutical composition.

The following are examples of the practice of the invention. They are not to be construed as limiting the scope of the invention in any way.

### EXAMPLES

Example 1

### Abrogation of Hydrogen Peroxide-Induced c-Abl Activity by STI571

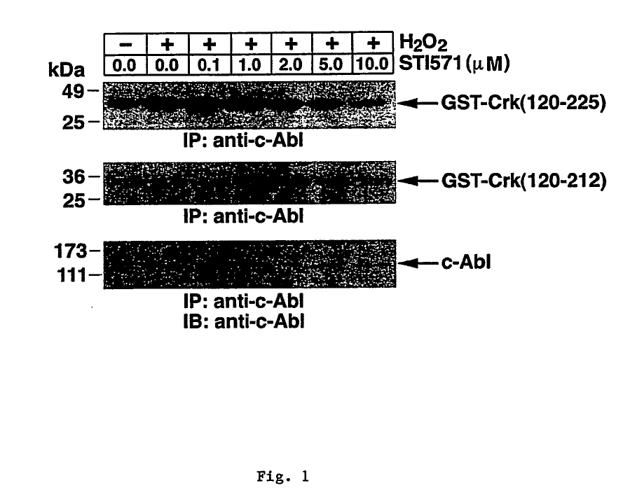

To determine whether STI571 affects reactive oxygen species (ROS)-induced signaling, mouse embryo fibroblasts (MEFs) were pretreated with 0.1 to 10 μM STI571 for 24 hours and then exposed to hydrogen peroxide (H₂O₂) for 15 minutes. Cell lysates were subjected to immunoprecipitation (IP) with an anti-c-Abl antibody (24-11; Santa Cruz Biotechnology) as described (Sun et al. (2000) J. Biol. Chem. 275:17237-40). The immunoprecipitates were incubated with [γ-³²P]ATP and GST-Crk(120-225) (FIG. 1, upper panel) or [γ-³²P]ATP and GST-Crk(120-212) (FIG. 1, middle panel) for 20 minutes at 30° C. as described (Sun et al. (2000) J. Biol. Chem. 275:17237-40). The reaction products were analyzed by SDS-PAGE and autoradiography to determine the ability of the immunoprecipitates to induce the phosphorylation of the GST-Crk substrates. The phosphorylation assay was used to detect the kinase activity of c-Abl and measure the effects of H₂O₂ and STI571 on this activity. The immunoprecipitates were also subjected to immunoblotting (IB) with the anti-c-Abl antibody (24-11) (FIG. 1, lower panel).

The results demonstrated that c-Abl is activated in response to H₂O₂ treatment (FIG. 1, upper panel, lane 2). Pretreatment with 0.1 and 1.0 μM STI571 had little effect on H₂O₂-induced c-Abl activity, while 2 and 5 μM STI571 pretreatment partially blocked c-Abl activation (FIG. 1, upper panel, lanes 3-6). Pretreatment of MEFs with 10 μM STI571 was associated with a c-Abl activity lower than the constitutive level of activity found in MEFs exposed to neither H₂O₂ nor STI571 (see FIG. 1, upper panel, lane 7). As a control, no phosphorylation was detected when the anti-c-Abl immunoprecipitates were incubated with GST-Crk(120-212), a peptide lacking the c-Abl phosphorylation site at Tyr-221 (FIG. 1, middle panel). Immunoblot analysis of the anti-c-Abl immunoprecipitates demonstrated the presence of equal amounts of c-Abl protein among the various treatments (FIG. 1, lower panel). These findings demonstrate that activation of c-Abl in the oxidative stress response is abrogated by 10 μM STI571.

### Example 2

### STI571 Blocks Hydrogen Peroxide-Induced Mitochondrial Targeting of c-Abl

To determine whether mitochondrial targeting of c-Abl in the ROS response is modulated by STI571, the subcellular localization of c-Abl was investigated by immunofluorescence microscopy.

MEFs were treated with 10 μM STI571 for 24 hours and then exposed to 1 mM H₂O₂ for 1 hour. After washing, the cells were fixed and incubated with an anti-c-Abl antibody (K-12; Santa Cruz) followed by Texas Red-conjugated goat anti-rabbit IgG as described (Kumar et al. (2001) J. Biol. Chem. 17281-85). Mitochondria were stained with 100 nM Mitotracker° Green (Molecular Probes, Eugene, Oreg.). Nuclei were stained with 1 μg/ml 4,6-diamino-2-phenylindole (DAPI). Cells were visualized by digital confocal immunofluorescence. Images were captured with a CCD camera mounted on a Zeiss Axioplan 2 microscope. Images were deconvoluted using SlideBook™ software as described (Kumar et al. (2001) J. Biol. Chem. 17281-85).

Examination of fluorescence markers in untreated MEFs showed distinct patterns for c-Abl (red signal) and the mitochondrion-selective dye (green signal). In concert with the localization of c-Abl to mitochondria, exposure of MEFs to H₂O₂ resulted in a redistribution of the fluorescence signals (red+green→yellow/orange). In contrast, the pretreatment of MEFs with STI571 blocked H₂O₂-induced mitochondrial targeting of c-Abl.

To confirm the effects of STI571, mitochondrial fractions from the treated MEFs were analyzed by immunoblotting with an anti-c-Abl antibody. Mitochondrial fractions were prepared as described (Kumar et al. (2001) J. Biol. Chem. 17281-85) and subjected to immunoblotting with anti-c-Abl, anti-HSP60 (Stressgen, Va., British Columbia), anti-p-actin (Sigma), or anti-PCNA (Calbiochem). Whole cell lysate (WCL) was included as a control. Antigen-antibody complexes were visualized by enhanced chemiluminescence. The intensity of the signals was determined by densitometric scanning. At 1 and 2 hours after H₂O₂ exposure, a greater than 5-fold increase in mitochondrial c-Abl protein was detected in those MEFs exposed to H₂O₂ alone, as compared to those MEFs that were exposed to H₂O₂ and treated with STI571. These experiments further demonstrated that STI571 blocked H₂O₂-induced targeting of c-Abl to mitochondria. Immunoblotting for the mitochondrial HSP60 protein demonstrated equal loading of the lanes. Purity of the mitochondrial fractions was confirmed by reprobing the blots with antibodies against the cytoplasmic β-actin protein and the proliferating cell nuclear antigen (PCNA).

The ROS-induced mitochondrial targeting of c-Abl was also found to be blocked by STI571 in H₂O₂-treated human U-937 myeloid leukemia cells as well as in human neuroblastoma cells, indicating that the inhibitory effect occurs in diverse cell types.

### Example 3

### STI571 Attenuates Hydrogen Peroxide-Induced Loss of Mitochondrial Transmembrane Potential

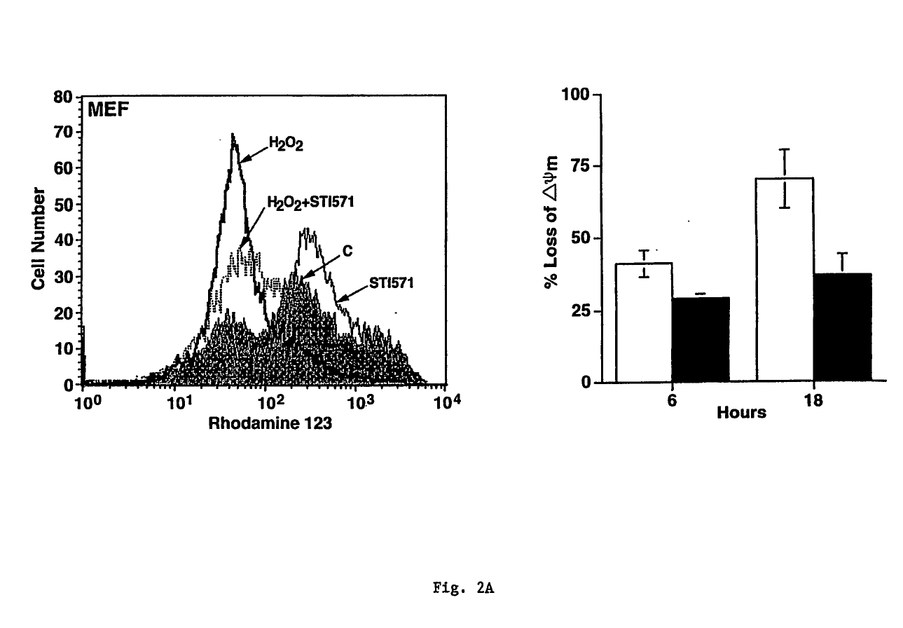

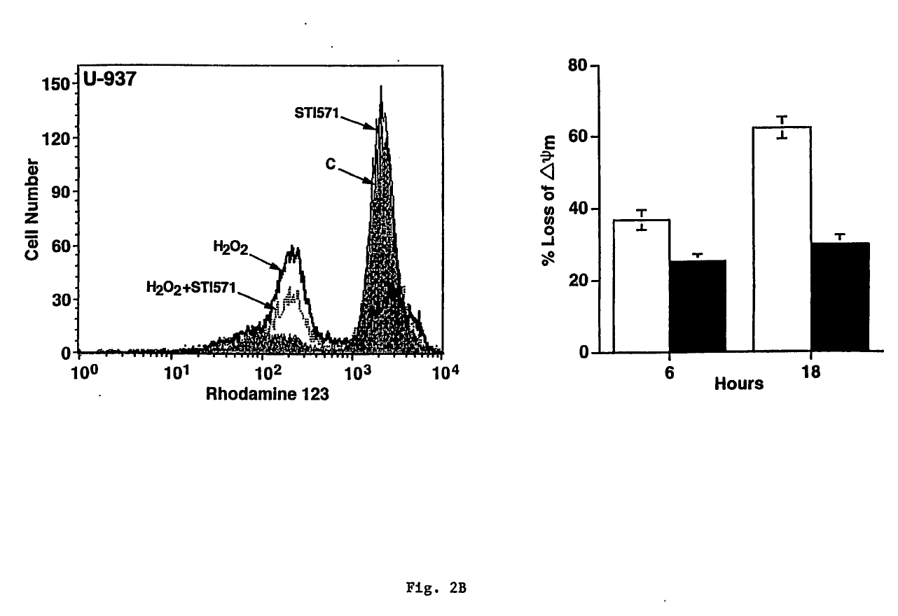

To assess the involvement of c-Abl in ROS-induced decreases in mitochondrial transmembrane potential, MEFs (FIG. 2A) and U-937 cells (FIG. 2B) were treated with 10 μM STI571 for 24 hours and then exposed to 1 mM H₂O₂ for 6 hours. The cells were stained with 50 ng/ml Rhodamine123 (Molecular Probes) for 15 minutes at 37° C. Samples were analyzed by flow cytometry using 488 nm excitation and measurement of emission through a 576/26 (ethidium) bandpass filter (FIGS. 2A and 2B, left panels). The percentage (mean±S.E.) of control mitochondrial transmembrane potential obtained after treatment with STI571 for 24 hours and then H₂O₂ for 6 or 18 hours was determined from three separate experiments (FIGS. 2A and 2B, right panels).

Flow cytometry analysis demonstrated that H₂O₂ induced a loss of mitochondrial transmembrane potential in MEFs (FIG. 2A, left panel). Treatment with STI571 alone had no apparent effect on mitochondrial transmembrane potential FIG. 2A, left panel). However, STI571 substantially inhibited the H₂O₂-induced decrease in mitochondrial transmembrane potential (FIG. 2A, left panel). The inhibitory effects of STI571 were also detectable at longer periods of H₂O₂ exposure (FIG. 2A, right panel). Similar findings were obtained when U-937 cells were pretreated with STI571 and then exposed to H₂O₂ (FIG. 2B). In FIGS. 2A and 2B, the black bars represent cell treated with H₂O₂ and STI571, and the white bars represent cells treated with H₂O₂ alone.

### Example 4

### STI571 inhibits the Apoptotic Response to Oxidative Stress

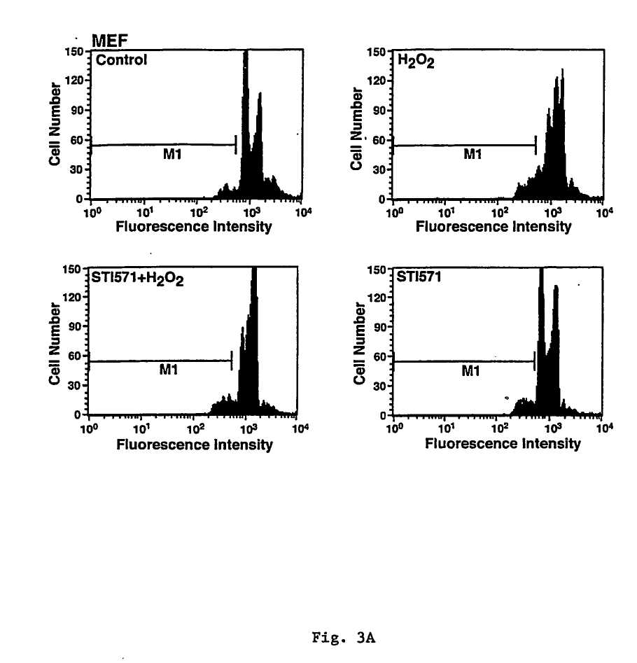

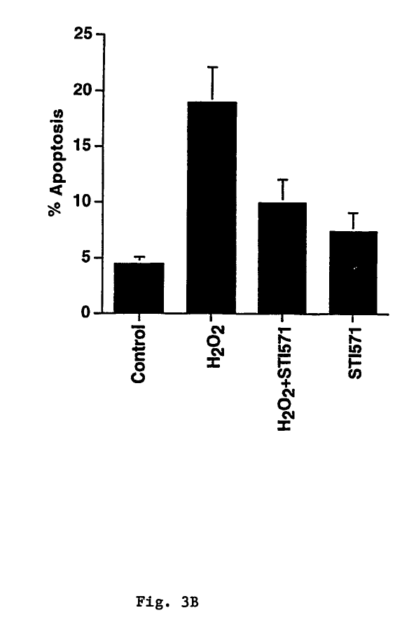

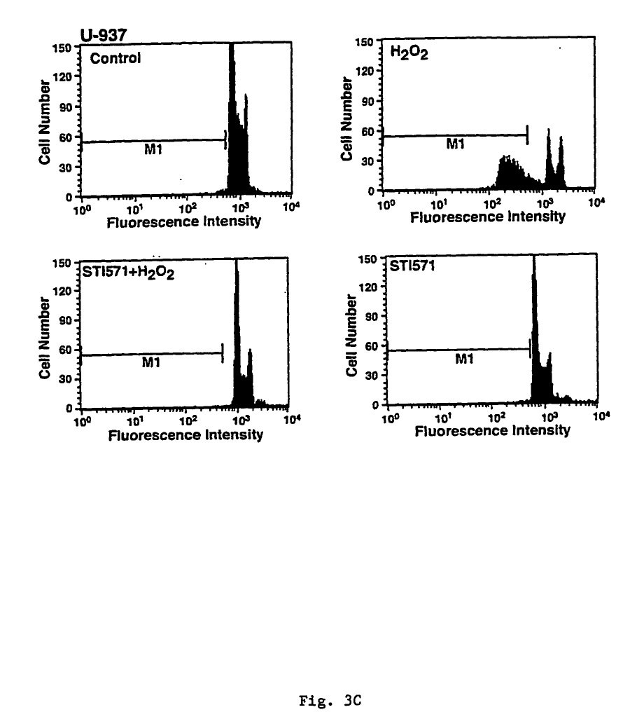

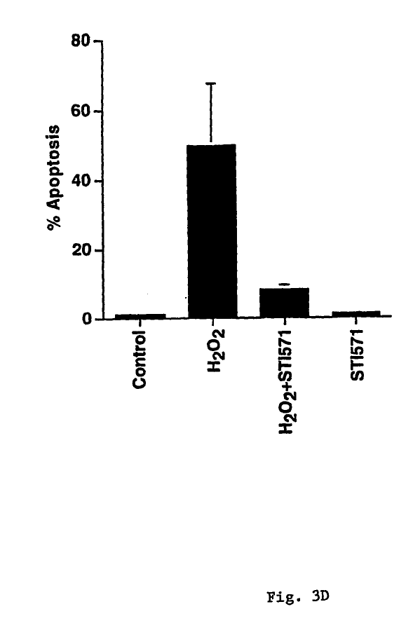

To assess the effects of STI571 on H₂O₂-induced apoptosis, MEFs (FIGS. 3A and 3B) and U-937 cells (FIGS. 3C and 3D) were treated with 10 μM STI571 for 24 hours and then exposed to 1 mM H₂O₂ for 18 hours. Ethanol-fixed cells were stained with propidium iodide and monitored for sub-G1 DNA by FACScan (Becton-Dickinson) (FIGS. 3A and 3C). The percentage (mean±S.E.) of cells with sub-G1 DNA was determined from three separate experiments (FIGS. 3B and 3D).

As shown in FIGS. 3A and 3B, MEFs exposed to H₂O₂ were induced to undergo apoptosis. Pretreatment of MEFs with STI571 was associated with a substantial block of H₂O₂-induced apoptosis (FIGS. 3A and 3B). STI571 decreased H₂O₂-induced apoptosis by 50% (FIGS. 3A and 3B). STI571 pretreatment also blocked the apoptotic response of U-937 cells to H₂O₂ (FIGS. 3C and 3D). STI571 inhibited H₂O₂-induced apoptosis of U-937 cells by over 80% (FIGS. 3C and 3D). These findings demonstrated that STI571 attenuates the apoptotic response to oxidative stress.

### Example 5

### ROS Induce the Formation of c-Abl-Arg Complexes

c-Abl forms molecular complexes with Arg, a nonreceptor tyrosine kinase that has an overall structure similar to that of c-Abl. To assess the effects of ROS on the formation of c-Abl-Arg complexes, lysates from H₂O₂-treated MCF-7 cells were subjected to immunoprecipitation with anti-c-Abl. Immunoblot analysis of the precipitates with anti-Arg demonstrated that exposure to 10 mM H₂O₂ had little if any effect on the association of c-Abl and Arg. In contrast, treatment with 40 and 160 mM H₂O₂ was associated with an increase in c-Abl-Arg complexes. Exposure to 640 mM H₂O₂ resulted in an association between c-Abl and Arg that was comparable to that found in control cells. Based on the total amount of Arg in lysates subjected to immunoprecipitation, approximately 1% of the Arg protein was complexed with c-Abl in control cells. Treatment with 40 mM H₂O₂ increased the formation of c-Abl-Arg complexes 3.2-fold, such that approximately 3% of the Arg protein was complexed with c-Abl.

To determine whether the interaction between c-Abl and Arg occurs in the response to agents other than H₂O₂ that induce oxidative stress, cells were treated with menadione (Sigma), a redox-cycling agent that increases ROS generation (Klohn et al. (1997) Chem-Biol Interactions 106:15-28). Similar to the case with H₂O₂, treatment with 25 mM menadione increased the formation of c-Abl/Arg complexes, while exposure to higher concentrations had less of an effect. Similar results were obtained when cells were treated with 20 ng/ml tumor necrosis factor a (TNFα) (Promega, Madison, Wis.) to induce an endogenous oxidative stress response (Chandel et al. (2001) J Biol Chem 276:42728-36; Schreck et al. (1991) EMBO J 10:2247-2258).

In studies with 293 cells expressing Flag-Arg, the constitutive level of c-Abl-Arg complexes was increased compared to undetectable levels found in wild-type cells. Treatment of the 293 cell transfectants with 160 mM H₂O₂ resulted in an increase in the association between c-Abl and Arg. As found in MCF-7 cells and MEFs, exposure to 640 mM had less of an effect. Analysis of the anti-Flag immunoprecipitates for phosphorylation of GST-Crk(120-225) demonstrated that H₂O₂ treatment induced activity of the c-Abl-Arg complex. To distinguish Arg from c-Abl, cells were transfected to express GFP-Arg. Immunoblot analysis of anti-GFP immunoprecipitates with anti-P-Tyr confirmed that H₂O₂ induced tyrosine phosphorylation of Arg. Similar results were obtained with expression of GFP-Arg(K-R), indicating that increased tyrosine phosphorylation is not due to Arg autophosphorylation. These findings demonstrate that ROS induce: the formation of c-Abl-Arg complexes; activation of the c-Abl-Arg complex; and tyrosine phosphorylation of Arg.

### Example 6

### c-Abl and Arg are Required for ROS-Induced Apoptosis

To assess involvement of c-Abl and Arg in the response of cells to ROS, MCF-7 cells were studied for H₂O₂-induced apoptosis. Compared to wild-type MCF-7 cells, treatment of MCF-7/Flag-Arg cells with 250 μM H₂O₂ was associated with an increase in sensitivity to ROS-induced apoptosis. In contrast, stable expression of Arg(K-R) in MCF-7 cells resulted in an attenuated apoptotic response. Similar findings were obtained in MCF-7 cells stably expressing c-Abl(K-R). In studies of MEFs, c-abl^{-/-} cells were less sensitive than wild-type cells to treatment with 40 or 250 μM H₂O₂. The finding that stable expression of c-Abl in the c-abl^{-/-} cells reconstitutes the apoptotic response to ROS demonstrated dependence on c-Abl. The arg^{-/-} cells were also less sensitive to ROS-induced apoptosis as compared to wild-type cells. To determine whether c-Abl and Arg are required for apoptosis in response to other inducers of oxidative stress, wild-type, c-Abl^{-/-} and Arg^{-/-} MEFs were treated with menadione. The results demonstrated that compared to wild-type MEFs, menadione-induced apoptosis is attenuated in c-Abl^{-/-} and Arg^{-/-} cells. Similar results were obtained when these cells were treated with TNFα and cyclohexamide (Sigma) to induce apoptosis (Johnson et al. (2000) J Biol Chem 275:31546-53).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of reducing or preventing oxidative stress-associated cell death, the method comprising:
selecting an individual diagnosed as having or being at risk of contracting a disorder characterized by excessive oxidative stress-associated cell death; and
administering to the individual a composition comprising an N-phenyl-2-pyrimidine-amine in an amount effective to reduce or prevent oxidative stress-associated cell death in the individual.

2. The method of claim 1, wherein the N-phenyl-2-pyrimidine-amine is 4-[(4-Methyl-1-piperazinyl)methyl]-N-[4-methyl-3-[[4-(3-pyridinyl)-2-pyrimidinyl]amino-]-phenyl]benzamide methanesulfonate.

3. The method of claim 2, wherein the individual has been diagnosed as having a disorder characterized by excessive oxidative stress-associated cell death.

4. The method of claim 2, wherein the individual has been diagnosed as being at risk of contracting a disorder characterized by excessive oxidative stress-associated cell death.

5. The method of claim 2, wherein the individual has been diagnosed as having a neurological disorder.

6. The method of claim 5, wherein the neurological disorder is Alzheimer's disease, Parkinson's disease, Huntington's disease, amyotrophic lateral sclerosis, multiple sclerosis, retinitis pigmentosa, or spinal muscular atrophy.

7. The method of claim 6, wherein the individual has not been diagnosed as having chronic myelogenous leukemia

8. The method of claim 6, wherein the individual has not been diagnosed as having a cancer.

9. The method of claim 5, further comprising administering to the individual a second therapeutic compound, wherein the second therapeutic compound reduces or prevents symptoms of the neurological disorder.

10. The method of claim 9, wherein the second therapeutic compound is riluzole, tacrine, donepizil, carbidopa/levidopa, carbidopa/levidopa sustained release, pergolide mesylate, bromocriptine mesylate, selgiline, amantadine, or trihexyphenidyl hydrochloride.

11. The method of claim 9, wherein the second therapeutic compound is a dopamine receptor antagonist.

12. The method of claim 9, wherein the second therapeutic compound is a glutamate excitotoxicity inhibitor, growth factor, nitric oxide synthase inhibitor, cyclo-oxygenase inhibitor, ICE inhibitor, neuroimmunophilin, N-acetylcysteine, procysteine, antioxidant, or lipoic acid.

13. The method of claim 5, further comprising carrying out a neurological test on the individual after administering the composition to the individual.

14. The method of claim 13, further comprising again administering the composition to the individual after carrying out the neurological test, wherein the amount of the composition administered in the second administration is determined at least in part based upon results obtained from the neurological test.

15. The method of claim 2, wherein the individual has been diagnosed as being at risk of contracting a neurological disorder.

16. The method of claim 15, wherein the neurological disorder is Alzheimer's disease, Parkinson's disease, Huntington's disease, amyotrophic lateral sclerosis, multiple sclerosis, retinitis pigmentosa, or spinal muscular atrophy.

17. The method of claim 2, wherein the disorder is caused by an ischemia/reperfusion injury.

18. The method of claim 17, wherein the individual has been diagnosed as having had a myocardial infarction or stroke.

19. The method of claim 17, wherein the individual has undergone or is undergoing an organ transplant surgery.

20. The method of claim 17, wherein the individual has undergone or is undergoing coronary bypass surgery.

21. The method of claim 17, further comprising administering to the individual a second therapeutic compound, wherein the second therapeutic compound reduces or prevents symptoms of the disorder.

22. The method of claim 21, wherein the second therapeutic compound is a thrombolytic or an anticoagulant.

23. The method of claim 17, further comprising carrying out a test for ischemia/reperfusion injury on the individual after administering the composition to the individual.

24. The method of claim 23, further comprising again administering the composition to the individual after carrying out the test for ischemia/reperfusion injury, wherein the amount of the composition administered in the second administration is determined at least in part based upon results obtained from the test for ischemia/reperfusion injury.

25. The method of claim 2, wherein the individual has been diagnosed as having an inflammatory disorder.

26. The method of claim 25, wherein the inflammatory disorder is arthritis.

27. The method of claim 2, further comprising evaluating the viability of a neurological or cardiovascular tissue of the individual following the administration of the composition to the individual.

28. The method of claim 2, wherein the composition is administered to the individual by injection.

29. The method of claim 2, wherein the composition is administered to the individual via a catheter.

30. A method of preventing or reducing cell death in a cell population, the method comprising:
providing a cell population; and
contacting the cell population with a composition comprising an N-phenyl-2-pyrimidine-amine in an amount effective to prevent or reduce cell death in the cell population.

31. The method of claim 30, further comprising determining the viability of the cell population, wherein the viability of the cell population is increased as compared to the viability predicted in the absence of contacting the cell population with the composition.

32. The method of claim 31, wherein the N-phenyl-2-pyrimidine-amine is 4-[(4Methyl-1-piperazinyl)methyl]-N-[4methyl-3-[[4(3-pyridinyl)-2-pyrimidinyl]amino-]-phenyl]benzamide methanesulfonate.

33. The method of claim 32, wherein the cell population does not comprise cancer cells.

34. The method of claim 32, wherein the cell population does not comprise chronic myelogenous leukemia cells.

35. The method of claim 33, wherein the cell population comprises neural cells.

36. The method of claim 33, wherein the cell population comprises cells that have undergone an ischemia/reperfusion injury.

37. A method of reducing or preventing aging-related cellular degeneration in an individual, the method comprising administering to the individual a composition comprising an N-phenyl-2-pyrimidine-amine in an amount effective to reduce or prevent aging-related cellular degeneration in the individual.

38. The method of claim 37, wherein the N-phenyl-2-pyrimidine-amine is 4-[(4-Methyl-l-piperazinyl)methyl]-N-[4-methyl-3-[[4-(3-pyridinyl)-2-pyrimidinyl]amino-]-phenyl]benzamide methanesulfonate.

39. The method of claim 37, wherein the individual has not been diagnosed as having chronic myelogenous leukemia

40. The method of claim 37, wherein the individual has not been diagnosed as having a cancer.

41. A kit comprising a composition comprising an N-phenyl-2-pyrimidine-amine and written instructions for use to reduce or prevent aging-related cellular degeneration or treat a disorder characterized by excessive oxidative stress-associated cell death.

42. The kit of claim 41, wherein the written instructions are for use to treat a neurological disorder.

43. The kit of claim 41, wherein the written instructions are for use to treat a disorder caused by an ischemia/reperfusion injury.

44. The kit of claim 41, wherein the N-phenyl-2-pyrimidine-amine is 4-[(4-Methyl-1-piperazinyl)methyl]-N-[4-methyl-3-[[4-(3-pyridinyl)-2-pyrimidinyl]amino-]-phenyl]benzamide methanesulfonate.

45. A pharmaceutical composition comprising an N-phenyl-2-pyrimidine-amine and a second therapeutic compound that is effective for the treatment of a disorder characterized by excessive oxidative stress-associated cell death.

46. The pharmaceutical composition of claim 45, wherein the disorder is a neurological disorder.

47. The pharmaceutical composition of claim 45, wherein the N-phenyl-2-pyrimidine-amine is 4-[(4-Methyl-1-piperazinyl)methyl]-N-[4-methyl-3-[[4-(3-pyridinyl)-2-pyrimidinyl]amino-]-phenyl]benzamide methanesulfonate.
